(19) United States
(12) Patent Application Publication
(10) Pub. No.: US 2019/0128817 A1
(43) Pub. Date: May 2, 2019

(54) HYDROCARBON DETECTION

(71) Applicant: POLARCUS DMCC, Dubai (AE)

(72) Inventor: Erik GODØY, Nesbru (NO)

(21) Appl. No.: 16/092,222

(22) PCT Filed: May 15, 2017
(86) PCT No.: PCT/NO2017/050118

§ 371 Date: Oct. 9, 2018

(30) Foreign Application Priority Data
May 24, 2016 (NO) 20160882

(51) Int. Cl. G01N 21/85; G01S 17/02; G01N 33/18; G01S 7/48; G01S 17/89; H04L 9/06; G06F 21/60

(52) U.S. Cl. CPC G01N 21/85; G01S 17/023; G01N 33/1833; G06F 21/602; G01S 17/89; H04L 9/0643; G01S 7/4802

(57) ABSTRACT

The invention concerns a system (100) for detecting pollution (2) on or at a sea surface (3), wherein the system (100) is adapted for mounting on a marine vessel (1). The system comprises a detector (110) with a LIDAR for providing digital pollution data (112) relating to pollution (2) in a region (40) along a path (4) travelled by the marine vessel (1); a positioning system (120) for providing positioning data; a clock (131) for providing a digital timestamp; and a computer (130) for collecting, combining and storing digital data (141). The computer (130) is configured to execute a secure hashing algorithm (133) on pollution data (112) with associated positioning data and a timestamp to produce a digest, execute an encryption function (134) on the digest using a private key (sk) and to store the data (141) and encrypted digest (142) in a database (140).

### FIELD OF THE INVENTION

The present invention concerns a system for remote detection of pollution on or at a sea surface.

### PRIOR AND RELATED ART

Oil spills from fixed and floating production facilities, pipelines and vessels, e.g. vessels cleaning their bunker tanks at sea, may harm birds and marine life and pollute shores. Other pollution include solid synthetic objects, e.g. ropes, destroyed or abandoned fishing nets or synthetic rubber particles from vehicle tyres. Some objects, e.g. ropes, may be entangled in a propeller, and are a direct threat to ship traffic. Other objects, e.g. synthetic rubber particles, degrade slowly and are primarily harmful to birds and marine life offshore and onshore.

LIDARs (LIght Detection And Ranging) are commonly used detectors for remote detection of oil spills etc. Suitable laser sources emit light in the near UV-range, e.g. 300-400 nm, which has a sufficient energy to excite molecules in the oil spill. The molecules emit fluorescent light when they return to a less excited state, and the resulting fluorescence is characteristic for the type and concentration of compounds in the oil spill. Thus, a spectrum showing intensities of fluorescence at different wavelengths is a ‘fingerprint’ for the oil spill.

A LIDAR may output in the time domain instead of or in addition to the spectral output described above. A temporal output is useful for ranging, i.e. determining the distance to an object. Laser light with wavelength in the range 300-400 mm penetrates water sufficiently to detect objects under water.

Hyperspectral imagining involves acquiring images using several narrow spectral bands to create a data cube. For example, a two dimensional array of pixels may record several images of one scene, each image containing information acquired at a limited range of wavelengths. In hyperspectral laser induced fluorescence imaging, the recorded scene is ‘viewed’ in the fluorescent light created by the source laser. Hence, the scene is dark when viewed at wavelengths with little or no fluorescence, and brightly lit at frequencies where the fluorescence is intense. There are several common ways to acquire images for the hyperspectral data cube, including spatial scanning, spectral scanning and spatiospectral scanning, each with known advantages and shortcomings.

In continuous monitoring, several images from a LIDAR is acquired each second at a fixed rate. The mathematics associated with managing pixels changing from image to image is well understood, and implemented in current graphic cards or GPUs. Thus, a GPU typically facilitates the task of identifying the extent of an oil spill or a solid object.

LIDARs of different types with different properties and post processors are commercially available and need no further description herein.

Current environmental surveys for offshore areas often involve airplanes, drones and/or satellites, all of which require relatively expensive equipment, e.g. sensitive LIDARs capable of detecting weak return signals. Thus, current environmental surveys are relatively expensive, and not performed as often as desirable.

A main objective of the present invention is to provide a system that provides environmental pollution data for an area at sea at lower cost than present methods.

### SUMMARY OF THE INVENTION

This is achieved by a system according to claim 1. Further features and benefits appear in the dependent claims 2-10.

More particularly, the invention concerns a system for detecting pollution on or at a sea surface, wherein the system is adapted for mounting on a marine vessel. The system comprises a detector with a LIDAR for providing digital pollution data relating to pollution in a region along a path travelled by the marine vessel; a positioning system for providing positioning data; a clock for providing a digital timestamp; and a computer for collecting, combining and storing digital data. The computer is configured to execute a secure hashing algorithm on pollution data with associated positioning data and a timestamp to produce a digest, execute an encryption function on the digest using a private key and to store the data and encrypted digest in a database.

The terms “on or at a sea surface” and “region” include a water column below the surface. A system designed for a marine vessel can use a less sensitive detector than systems designed for e.g. an airplane or a satellite due to a significantly shorter distance from the detector to the sea surface. Alternatively, the SNR, the spectral resolution, the temporal resolution and/or the spatial resolution can be improved compared to those obtainable from, for example, an airplane with a similar detector. The improved data quality enables more precise spectral characterisation of an oil spill and hence of its source. The precise characteristic may be used as evidence, e.g. for charging fines. The improved data quality also enables detection of synthetic material that may harm sea birds and marine life.

The detector comprises a LIDAR and a postprocessor for processing data acquired by the LIDAR and presenting a desired output, e.g. the extent and nature of an oil spill or a spatial distribution of solid objects such as a synthetic rope or particles from car tires.

The secure hashing algorithm ensures that nobody can alter the pollution data or its associated position, date and time without detection. The subsequent encryption with a private key authenticates the origin of the data as long as the private key remains secret. Any recipient with access to a public key associated with the private key can decrypt the encrypted digest, compute a hash from the data and compare the decrypted digest with the computed hash. If they match, the recipient is confident that the signer had access to the private key, and that nobody has altered the data.

The term “database” should be construed broadly, and is intended to include any collection of pollution data stored in a digital storage, e.g. files in a file system and/or data in a relational database.

In a preferred embodiment, the marine vessel detects pollution as a secondary task. For example, a merchant vessel transporting goods between ports on different continents may collect environmental data along an international trade route at little additional cost. Similarly, a vessel in regular traffic along a coast line may discover, record and characterise a discharge from a vessel that has cleaned its bunker tanks at sea. In a third example, the marine vessel is a seismic survey vessel that systematically covers an area of hundreds or thousands of square kilometres during a survey. These and other vessels may collect environmental data with spatial, temporal and spectral resolutions that a satellite cannot achieve. A drone flying low might provide data with a similar quality, but the acquisition cost would be forbidding.

In accordance with the above, the detector may provide a spectral output for characterising a hydrocarbon mixture. The spectral output shows a peak intensity at wavelengths emitted from different components in the hydrocarbon mixture. Thus, a later analysis may at least limit the number of possible sources and possibly identify the source conclusively. In addition, a similar digitally signed spectral output obtained from the marine vessel's own bunker oil may provide undeniable evidence that the marine vessel was not the source of an oil spill.

The system may further comprise a lookup table and/or an algorithm for determining the age of the hydrocarbon mixture. If the age of an oil spill can be determined to, for example, an interval of a few days, the source may be limited to vessels that were present in an area in the interval compensated for wind, waves and current. Some oil spills, e.g. from a production well, contain a broad range of hydrocarbons and typically a significant amount of sulphur. Lighter components, e.g. such as aliphates up to about C10 and volatile aromatic compounds, evaporate more easily than heavier components. Thus, the altered relative composition may be used to estimate the age for some oil spills.

In addition or alternatively, the lookup table and/or algorithm may include parameters for mechanical decomposition of the oil spill. This is particularly useful for spills of marine fuels, i.e. bunker oil. Marine fuels typically contain little sulphur due to emission standards in force since the 1970s, and comprise heavier components. Bunker oil is a residue after naphtha, kerosene and other light qualities have been removed, so relative concentrations of lighter components cannot determine the age of bunker oil. However, an oil spill is likely to be broken up into smaller patches spread over a larger area due to wind, waves and currents. Thus, patches of similar oil spread over a large area may be used to estimate the age and original location of the oil spill.

The detector may provide a distance to and extension of an object in the region around the path of the marine vessel. For this, a LIDAR provides temporal data. The LIDAR providing data in the time domain may be the same unit as the LIDAR providing a spectral output. The postprocessor associated with the detector may implement a fast Fourier transform algorithm to convert from the time domain to the frequency domain and vice versa.

In some embodiments, the detector comprises a digital filter for providing a concentration of solid objects in a scanned region. Due to the improved SNR and spatial resolution, the solid objects may be relatively small particles, and the digital pollution data could be a count of particles per volume unit. A skilled person may implement such a filter using high-level functions implemented in commercial GPUs.

In some embodiments, the detector comprises a hyperspectral laser induced fluorescence LIDAR. The associated postprocessor may slice the acquired data cube in different ways to provide spectral, temporal or spatial output data.

In all embodiments, a preferred LIDAR has a laser light source emitting in the range 300-400 nm. As noted above, these wavelengths are sufficiently short to excite hydrocarbon molecules for a fluorescent spectrum. The wavelengths in this range also penetrate water to detect solid objects in a water column below the surface. The maximum depth depends on the angle of view, the attenuation of the selected wavelength and the water quality.

The database may be configured to receive digitally signed data from several marine vessels. A national database might contain mandatory spectral signatures of the bunker oil of all vessels entering and/or leaving a national port. This would facilitate identification of oil spills, and might prevent vessels from cleaning bunker tanks in open seas. An international database containing data from merchant ships, survey vessels etc. would be a valuable tool for identifying and classifying pollution, for cleaning up and/or for determining trends regarding oil contamination and synthetic solid particles in the oceans.

### BRIEF DESCRIPTION OF THE DRAWINGS

The invention will be explained in greater detail by means of examples with reference to the accompanying drawings, in which:

FIG. 1 illustrates an environmental survey according to the invention, and

FIG. 2 illustrates a system according to the invention.

### DETAILED DESCRIPTION

The drawings are intended to illustrate the principle of the invention, and are not to scale. Numerous details known to those skilled in the art are omitted for clarity.

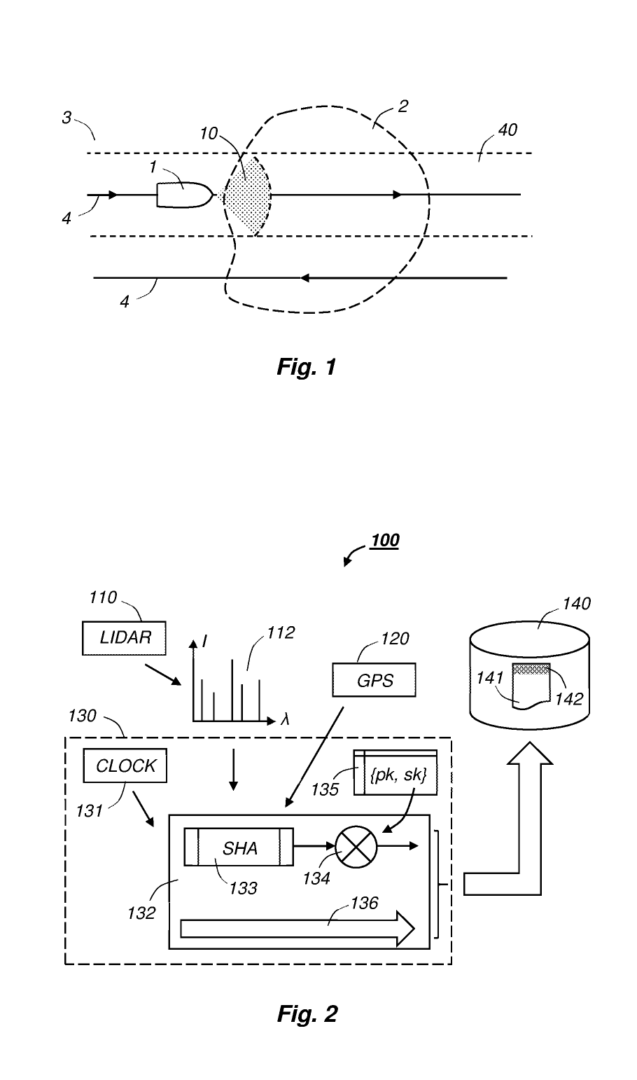

FIG. 1 shows a marine vessel 1 approaching a polluted area 2 on a sea surface 3. The marine vessel may perform some unrelated task, e.g. a seismic survey, such that the present environmental survey is a bonus at little additional cost. As shown, the vessel 1 travels along a path 4 that might be followed by a seismic survey vessel. However, the path 4 might be any path followed by a merchant ship, cruise ship or other marine vessel over an ocean or along a coast. A detector aboard the marine vessel 1 scans a sector 10, e.g. in front of the vessel. Thus, the marine vessel 1 detects pollution on or at the sea surface 3 in a region 40 along the predetermined path 4.

The region 40 may include a water column below the sea surface 3 to identify hydrocarbons and solid objects at, as opposed to on, the sea surface 3. The actual visibility illustrated by the arc of sector 10 may be defined as the limit where the SNR drops below a useful level. Specifically, wavelengths around 420 nm, i.e. in the violet part of the visible spectrum, are least attenuated in seawater. Laser light in the near UV-spectrum have slightly shorter wavelengths. The human eye is most sensitive at slightly longer wavelengths in the blue spectrum, where the Sun radiates most of its energy. Clean seawater appears clear to both the laser and the human eye as it has a visibility of tens of meters. Dissolved organic material cause a yellow tint that reduces visibility. Phytoplankton in the ocean or particles near a coastline increase the noise, and may easily reduce the visibility to a few metres.

FIG. 2 illustrates a system 100 with a LIDAR 110 providing pollution data 112. Specifically, the shown pollution data 112 are spectral data indicating peak intensities at certain wavelengths λ of fluorescent light. The spectrum indicates type and possibly age of an oil spill, and is supplied to a computer 130.

A global positioning system 120, e.g. the American Navstar GPS or the Russian GLONASS, provides positioning data to determine the position and extension of the polluted area 2, and a clock 131 provides a timestamp, i.e. date and time, for a recording.

The pollution data, positioning data and timestamp is a large lump of data. Thus, it would have to be split into many smaller blocks for digital signing. Instead, the data are first hashed by a predetermined secure hashing algorithm (SHA) 133 running in a processor 132. Suitable hashing algorithms are standardised, e.g. in the Internet transport layer security (TSL) protocols, and are thus publicly available. Hashing is much faster than signing, and may provide a digest with a block length adapted to a chosen block cipher.

The digest is further encrypted in the process 134, also within the processor 132. The encryption is performed using a secret or private key sk fetched from a secure internal storage 135. The private key sk is part of a cryptographic pair of keys {pk, sk} that are mathematically connected such that a message encrypted with sk can only be decrypted with a corresponding public key pk. The public key pk enables anyone to verify that a sender knows the secret sk.

The arrow 136 illustrates that the pollution data, positioning data and timestamp passes through the processor 132 parallel to the hashing function 133 and the encryption function or cipher 134. The cipher 134 is preferably standardised, c.f. the TSL protocols.

The pollution data, positioning data and timestamp are stored in a database 140, e.g. in a document 141 along with the encrypted digest 142. Later, any recipient with access to the public key pk can decrypt the block 142 to obtain a decrypted hash and compute a hash from the pollution data, positioning data and timestamp stored in the document 141. If the decrypted hash matches the computed hash, the recipient knows that the data originates from one knowing the corresponding private key sk.

Moreover, the hash is very sensitive to changes in the input. Thus, the recipient can rest assured that nobody has altered the data if the hash is unaltered. In addition to prove origin of data and integrity, hash-then-encrypt algorithms are secure against several known cryptographic attacks.

An additional benefit is that the marine vessel may be able to prove that it is not the source of an oil spill. For example, a sample of bunker oil used by the survey vessel may be characterised, digitally signed and stored as described above. Because any tampering with measurement data will be detected due to the properties of the secure hash algorithm 133, a mismatch between the bunker oil spectrum and the oil spill spectrum proves that the oil spill is not caused by bunker oil from the survey vessel.

In addition or as an alternative to oil spill detection, the system 100 may be configured to detect solid objects, e.g. ropes, fishing nets etc. in or at the sea surface 3. This may involve using a different LIDAR 110 providing output 112 in the time domain and possibly using a laser emitting at a different wavelength. The remaining parts of the system, i.e. the GPS 120, the computer 130 and database 140 with associated components, data and processes work in the manner described above. Embodiments detecting solid objects may also warn of harmful objects, e.g. a rope that may be entangled in a propeller.

The database 140 may be local to the marine vessel 1, i.e. only contain data acquired and signed on the marine vessel 1. In an alternative embodiment, the signed data are sent to a central database 140 managed by a national authority. Such a national database might contain mandatory spectral signatures of the bunker oil of all vessels entering and/or leaving a national port. This would facilitate identification of oil spills, and might prevent vessels from cleaning bunker tanks in open seas. In a third embodiment, the database is managed by an international authority, and contains data from merchant ships, survey vessels etc. Such an international database would be a valuable tool for identifying and classifying pollution, for cleaning up and/or for determining trends regarding oil contamination and synthetic solid particles in the oceans. There is no conflict between a local, a national and an international database, and data may be transferred between databases at different levels as desired.

While the invention has been described by examples, the skilled person will know many obvious adaptations and alternatives. The scope of the invention is defined by the following claims.

1-10. (canceled)

11. A system for detecting pollution on or at a sea surface, wherein the system is adapted for mounting on a marine vessel and comprises:
a detector with a LIDAR for providing digital pollution data relating to pollution in a region along a path travelled by the marine vessel;
a positioning system for providing positioning data;
a clock for providing a digital timestamp; and
a computer for collecting, combining and storing digital data, wherein the computer is configured to execute a secure hashing algorithm on the pollution data with associated positioning data and a timestamp to produce a digest, execute an encryption function on the digest using a private key and store the digital data and encrypted digest in a database.

12. The system according to claim 11, wherein the marine vessel is configured to detect pollution as a secondary task.

13. The system according to claim 11, wherein the detector is configured to provide a spectral output for characterising a hydrocarbon mixture.

14. The system according to claim 13, further comprising a lookup table and/or an algorithm for determining the age of the hydrocarbon mixture.

15. The system according to claim 14, wherein the lookup table and/or algorithm include parameters for mechanical decomposition of an oil spill.

16. The system according to claim 11, wherein the detector provides a distance to and extension of an object in the region around the path of the marine vessel.

17. The system according to claim 16, wherein the detector comprises a digital filter for providing a concentration of solid objects in a scanned region.

18. The system according to claim 11, wherein the detector comprises a hyperspectral laser induced fluorescence LIDAR.

19. The system according to claim 11, wherein the LIDAR has a laser light source emitting in the range 300-400 nm.

20. The system according to claim 11, wherein the database is configured to receive digitally signed data from several marine vessels.
